(19) United States
(12) Patent Application Publication
(10) Pub. No.: US 2025/0283860 A1
(43) Pub. Date: Sep. 11, 2025

(54) METHODS FOR CHROMATOGRAPHIC CHARACTERIZATION OF LIPID NANOPARTICLE COMPOSITIONS

(71) Applicant: Waters Technologies Corporation, Milford, MA (US)

(72) Inventors: Lavelay Kizekai, Coventry, RI (US); Abraham Finny, Marlborough, MA (US); Balasubrahmanyam Addepalli, Hopkinton, MA (US); Matthew Lauber, North Smithfield, RI (US)

(73) Assignee: Waters Technologies Corporation, Milford, MA (US)

(21) Appl. No.: 19/073,928

(22) Filed: Mar. 7, 2025

Related U.S. Application Data
(60) Provisional application No. 63/562,881, filed on Mar. 8, 2024.

(51) Int. Cl. G01N 30/88; G01N 30/34; G01N 30/56; G01N 30/74

(52) U.S. Cl. CPC G01N 30/88; G01N 30/34; G01N 30/56; G01N 30/74; G01N 2030/562; G01N 2030/8813

(57) ABSTRACT

The present disclosure is directed to methods for characterization of a sample by size exclusion chromatography (SEC), the sample including intact lipid nanoparticles (LNPs). The method generally includes loading the sample on a chromatographic column having an SEC packing material disposed therein, flowing a mobile phase through the SEC packing material to elute the intact LNPs, and detecting the eluted intact LNPs. The mobile phase includes an organic solvent, an aqueous buffer, and a non-ionic surfactant.

### RELATED APPLICATIONS

This application claims priority to and benefit of U.S. provisional application No. 63/562,881, filed Mar. 8, 2024, and entitled “Methods for Chromatographic Characterization of Lipid Nanoparticle Compositions.” The contents of U.S. provisional application No. 63/562,881 is incorporated herein by reference in its entirety.

### TECHNICAL FIELD

The present disclosure relates generally to chromatographic methods for the characterization of lipid nanoparticle compositions.

### BACKGROUND

Lipid nanoparticles (LNP) are valuable vehicles to deliver nucleic acids (e.g., siRNA, mRNA, and the like) as therapeutic agents and vaccines for the treatment or prevention of diseases and disorders, including viral infections and certain cancers. For example, the current COVID-19 mRNA vaccines from Pfizer-BioNTech and Moderna utilize LNPs to encapsulate mRNA, shield it from destructive enzymes, and shuttle it into cells. LNPs are generally prepared by encapsulating a nucleic acid with four lipid components (ionizable/cationic, phospholipid, cholesterol, and a pegylated lipid) through electrostatic interactions. To ensure potency and safety of LNP-based therapeutic agents and vaccines, their physical properties (size, molar mass), purity, and aggregation status must be accurately determined and documented. Due to the fragile nature, delicate structural integrity, and extreme differences in physicochemical properties of the various LNPs and their components, analytical characterization by techniques such as size exclusion chromatography (SEC) with multi-angle light scattering (MALS) detection pose major challenges such as poor-quality separation, poor sample recovery, column fouling, and contamination of flow cells in both optical and light scattering instruments. Accordingly, there is a need in the art to provide chromatographic methods for analysis of intact LNPs which provide high reproducibility, sample recovery, and efficiency and which overcome the noted challenges formerly associated with such methods.

### SUMMARY

The present technology is generally directed to methods for characterization of samples comprising intact lipid nanoparticles (LNPs). Such characterization may find utility in determining or confirming identity, purity, stability, and the like of LNPs, or in the process of developing of LNP-based therapeutic agents and vaccines. The methods generally feature performing chromatographic separations of intact LNPs in samples such as vaccines or drug products. This is in contrast to other analytical methods which comprise disrupting the LNPs and separating individual components thereof (e.g., various lipids and nucleic acid). The methods as disclosed herein utilize a unique mobile phase composition comprising an organic solvent, a non-ionic surfactant, and an aqueous buffer. The intact LNP is fully soluble in the mobile phase composition, thereby avoiding filtration effects (e.g., loss by precipitation) and providing complete and reproducible recovery of intact LNP without induction of artifacts. Surprisingly, the disclosed mobile phase formulation helps preserve the fragile structural integrity of delicate LNP samples and in some embodiments produces chromatograms that closely reflect the size variation or dispersity of the LNPs. Advantageously, linear response across injection volumes was consistently observed with this mobile phase composition, and LNP size analyses were consistent when measured by two orthogonal techniques using the disclosed mobile phase composition. In contrast, in the absence of the non-ionic surfactant, precipitation of LNPs, unwanted interactions with column stationary phase, frits, and/or detector flow cells was observed, resulting in poor separation, poor recoveries, and column fouling and contamination of flow cells in both optical and light scattering instruments.

Accordingly, in one aspect is provided a method for characterization of a sample comprising intact lipid nanoparticles (LNPs), wherein the characterization comprises performing size exclusion chromatography (SEC) on the sample, the method comprising:
a) loading the sample on a chromatography column including a compartment having interior walls defining wetted surfaces and containing a column packing material configured for SEC within said compartment;
b) flowing a mobile phase through the column packing material to elute the intact LNPs, the mobile phase comprising an organic solvent, an aqueous buffer, and a non-ionic surfactant, wherein the non-ionic surfactant is present in the mobile phase at a concentration sufficient to avoid denaturing the LNPs, minimize adsorption of the LNPs, and minimize interactions between the intact LNPs; and
c) detecting the eluted intact LNPs.

In another aspect is provided a method for characterization of a sample by size exclusion chromatography (SEC), the sample comprising intact lipid nanoparticles (LNPs), the method comprising:
a) loading the sample on a chromatographic device comprising a chromatography column including a compartment having interior walls defining wetted surfaces and containing an SEC packing material within said compartment;
b) flowing a mobile phase through the SEC packing material to elute the intact LNPs, the mobile phase comprising from about 1 to about 10% by volume of isopropanol, from about 90 to about 99% by volume of phosphate buffered saline, and from about 0.0001% to about 0.01% by volume of a hydroxy-terminated polyethylene oxide-polypropylene oxide copolymer; and
c) detecting the eluted intact LNPs.

The disclosure includes, without limitations, the following embodiments.

Embodiment 1: A method for characterization of a sample comprising intact lipid nanoparticles (LNPs), wherein the characterization comprises performing size exclusion chromatography (SEC) on the sample, the method comprising:
a) loading the sample on a chromatography column including a compartment having interior walls defining wetted surfaces and containing a column packing material configured for SEC within said compartment;
b) flowing a mobile phase through the column packing material to elute the intact LNPs, the mobile phase comprising an organic solvent, an aqueous buffer, and a non-ionic surfactant, wherein the non-ionic surfactant is present in the mobile phase at a concentration sufficient to avoid denaturing the LNPs, minimize adsorption of the LNPs, and minimize interactions between the intact LNPs; and
c) detecting the eluted intact LNPs.

Embodiment 2: The method of embodiment 1, wherein the non-ionic surfactant is present in an amount by volume from about 0.0001% to about 1%, based on the total volume of the mobile phase.

Embodiment 3: The method of embodiment 1, wherein the non-ionic surfactant is present in an amount by volume from about 0.0001% to 0.01%, based on the total volume of the mobile phase.

Embodiment 4: The method of embodiment 1, wherein the non-ionic surfactant is present in an amount by volume from 0.0005% to about 0.0015%, based on the total volume of the mobile phase.

Embodiment 5: The method of any one of embodiments 1-4, wherein the non-ionic surfactant is a hydroxy-terminated polyethylene oxide-polypropylene oxide copolymer.

Embodiment 6: The method of embodiment 5, wherein the non-ionic surfactant is a polyoxyethylene-polyoxypropylene block copolymer with the general formula (C₃H₆O·C₂H₄O)ₓ having a molecular weight of about 8400.

Embodiment 7: The method of any one of embodiments 1-6, wherein the organic solvent is isopropanol, acetonitrile, acetone, or a combination thereof.

Embodiment 8: The method of claim 1, wherein the organic solvent is isopropanol.

Embodiment 9: The method of embodiment 8, wherein the isopropanol is present in the mobile phase at a concentration from about 1% to about 10% (v/v).

Embodiment 10: The method any one of embodiments 1-9, wherein the aqueous buffer is phosphate buffered saline having a pH of about 7.4.

Embodiment 11: The method of embodiment 10, wherein the phosphate buffered saline and comprises from about 10 to about 100 mM sodium phosphate.

Embodiment 12: The method of embodiment 10 or 11, wherein the phosphate buffered saline comprises sodium chloride, potassium chloride, or a combination thereof.

Embodiment 13: The method of embodiment 12, wherein the phosphate buffered saline comprises:
from about 100 to about 500 mM sodium chloride, and from about 1 to about 10 mM potassium chloride; or
from about 100 to about 500 mM potassium chloride, and from about 1 to about 10 mM sodium chloride.

Embodiment 14: The method of any one of embodiments 1-13, wherein the mobile phase comprises the aqueous buffer in an amount by volume from about 90 to about 99%.

Embodiment 15: The method of any one of embodiments 1-14, wherein the mobile phase comprises about 95% phosphate buffered saline, about 5% isopropanol, and about 0.001% of hydroxy-terminated polyoxyethylene-polyoxypropylene triblock co-polymer.

Embodiment 16: The method of any one of embodiments 1-15, wherein the detecting is performed with a dual wavelength ultraviolet/visible detector, an evaporative light scattering detector, or a multi-angle light scattering (MALS) detector.

Embodiment 17: The method of any one of embodiments 1-16, wherein the detecting is performed with a dual wavelength ultraviolet/visible detector at a wavelength of 230, 260, or 280 nm.

Embodiment 18: The method of any one of embodiments 1-17, wherein at least a portion of the interior walls defining wetted surfaces comprises a coating configured to reduce hydrophobic secondary interactions between the intact LNPs and said interior walls.

Embodiment 19: The method of embodiment 18, wherein the coating includes a hydrophilic, non-ionic coating is the product of vapor deposited bis(trichlorosilyl)ethane or bis(trimethoxysilyl)ethane.

Embodiment 20: The method of any one of embodiments 1-19, wherein the packing material configured for SEC comprises diol-bonded porous particles having a particle size from about 1 μm to about 10 μm and an average pore diameter in a range from about 100 Å to about 5000 Å.

Embodiment 21: The method of any one of embodiments 1-20, wherein the packing material configured for SEC comprises particles with a siliceous surface, wherein the siliceous surface consists of a bonding phase formed by at least two silane compounds, wherein:
one of the at least two silane compounds is a dipodal hybrid silane comprising two indirectly linked silica atoms, and
one of the at least two silane compounds is a functionalized silane.

Embodiment 22: The method of embodiment 21, wherein the dipodal hybrid silane is selected from the group consisting of:

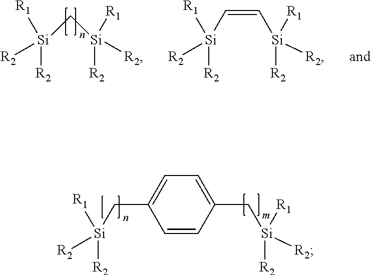

wherein:
R₁ is independently selected for each instance from chlorine, methoxy, and ethoxy;
R₂ is independently selected for each instance from alkyl, methoxy, ethoxy, and chlorine; and
n and m are each independently 1-4.

Embodiment 23: The method of embodiment 21 or 22, wherein the functionalized silane is selected from the group consisting of:

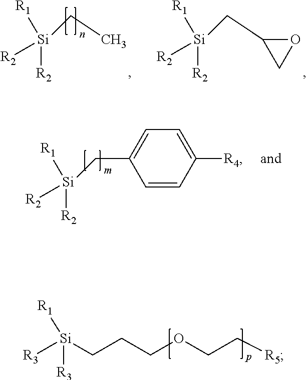

wherein:
R₁ is independently selected for each instance from chlorine, methoxy, and ethoxy;
R₂ is independently selected for each instance from alkyl, benzyl, methoxy, ethoxy, and chlorine;
R₃ is independently selected for each instance from alkyl, methoxy, ethoxy, and chlorine;
R₄ is H or benzene;
R₅ is hydroxy or methoxy;
n is 3, 7, or 17;
m is 0 or 1; and
p is an integer from 1-12.

Embodiment 24: A method for characterization of a sample by size exclusion chromatography (SEC), the sample comprising intact lipid nanoparticles (LNPs), the method comprising:
a) loading the sample on a chromatographic device comprising a chromatography column including a compartment having interior walls defining wetted surfaces and containing an SEC packing material within said compartment;
b) flowing a mobile phase through the SEC packing material to elute the intact LNPs, the mobile phase comprising from about 1 to about 10% by volume of isopropanol, from about 90 to about 99% by volume of phosphate buffered saline, and from about 0.0001% to about 0.01% by volume of a hydroxy-terminated polyethylene oxide-polypropylene oxide copolymer; and
c) detecting the eluted intact LNPs.

### BRIEF DESCRIPTION OF THE DRAWINGS

In order to provide an understanding of embodiments of the technology, reference is made to the appended drawings, which are not necessarily drawn to scale. The drawings are exemplary only and should not be construed as limiting the technology. The disclosure described herein is illustrated by way of example and not by way of limitation in the accompanying figures.

FIG. 1 is an overlay of two exemplary chromatographic separations of an intact LNP. One chromatogram is for a separation performed with a mobile phase according to a non-limiting embodiment of the disclosure, and one chromatogram is for a separation performed with a reference mobile phase (i.e., not including a non-ionic surfactant).

FIG. 2 is an overlay of three exemplary chromatographic separations of an intact LNP. One chromatogram is for a separation performed with a mobile phase according to a non-limiting embodiment of the disclosure, and the other two chromatograms are for separations performed with reference mobile phases (i.e., excluding a non-ionic surfactant; one with buffered saline only and one with buffered saline plus isopropanol).

FIG. 3, FIG. 4, and FIG. 5 show overlays of exemplary MALS spectra of LNPs according to the present technology. Each figure shows the MALS spectra of a Pfizer BioNTech Covid-19 vaccine (dashed line) and an empty LNP (solid line).

FIG. 3 shows the measured radius of gyration (R_{g}) of the vaccine (circles) and the empty LNP (triangles) at each elution time.

FIG. 4 shows the measured Rₕ of the vaccine (circles) and the empty LNP (triangles) at each elution time.

FIG. 5 shows the measured molar mass (in MDa) of the vaccine (circles) and the empty LNP (triangles) at each elution time.

### DETAILED DESCRIPTION

Before describing several example embodiments of the technology, it is to be understood that the technology is not limited to the details of construction or process steps set forth in the following description. The technology is capable of other embodiments and of being practiced or being carried out in various ways.

### Definitions

With respect to the terms used in this disclosure, the following definitions are provided. This application will use the following terms as defined below unless the context of the text in which the term appears requires a different meaning.

The articles “a” and “an” are used herein to refer to one or to more than one (i.e., to at least one) of the grammatical object of the article.

The term “lipid nanoparticle” or “LNP” as used herein refers to a nanoparticle composed of lipids and a nucleic acid payload. A lipid nanoparticle may comprise one or more types of lipids, including ionizable lipids (such as ionizable cationic lipids), phospholipids, structural lipids (such as cholesterol), and pegylated lipids. Lipids suitable for use in lipid nanoparticle compositions are further described in U.S. Pat. No. 11,786,607, PCT publication WO 2017/223,135, and U.S. Pat. No. 10,507,249 each of which are incorporated herein by reference.

The term “lipid” as used herein refers to a diverse group of organic compounds including fats, oils, and certain components of biological membranes which are characterized by a lack of appreciable interaction with water (i.e., exhibiting hydrophobicity). Lipids encompass molecules such as fatty acids and their derivatives (including tri-, di-, monoglycerides, and phospholipids), as well as other sterol-containing metabolites (e.g., cholesterol).

The term “nucleic acid” as used herein refers to linear biopolymer chains composed of series of nucleotides, which may also be referred to as “polynucleotides.” The term “nucleotide” as used herein refers to monomeric units consisting of three components: a 5-carbon sugar, a phosphate group, and a nitrogenous base. The chain length (number of nucleotides) of a nucleic acid may vary depending on, for example, the source and intended use. For example, a nucleic acid may comprise a relatively short chain (e.g., on the order of about 2 to about 20, or about 10 to about 200 nucleotides, generally referred to as oligonucleotides), or may comprise much larger chains (e.g., thousands, millions, or more nucleotides). Reference to a nucleic acid herein contemplates any nucleotide chain including, but not limited to, RNA, DNA, oligonucleotides, aptamers, and analogs or derivatives of any thereof, such as nucleotides comprising modified (i.e., artificial) bases or sugars. Further, reference to RNA and DNA include all forms thereof, such as may be present in a genome, chromosome, histone, or an isolated gene, those present either naturally or as artificially introduced into cells or viruses, those encoding genetic information or peptide sequences, and artificial, truncated, and/or fragments versions of any of the foregoing.

The term “intact” as used herein in reference to an intact LNP refers to a LNP which is in its native state and has not been subjected to any denaturing. In other words, the LNP has not been disrupted into the individual components (nucleic acid and lipids).

Embodiments of the present disclosure are now described in detail with the understanding that such embodiments are exemplary only. Such embodiments constitute what the inventors now believe to be the best mode of practicing the technology. Those skilled in the art will recognize that such embodiments are capable of modification and alteration.

As noted herein above, analytical characterization of intact LNPs by techniques such as size exclusion chromatography (SEC) pose challenges including poor-quality separation, poor sample recovery, column fouling, and contamination of flow cells. In view of these challenges, it would be desirable to provide chromatographic methods for analysis of intact LNPs which provide high reproducibility, sample recovery, and efficiency and which overcome the noted challenges formerly associated with such methods. According to the present disclosure, it has surprisingly been found that in some embodiments, SEC separations performed using a mobile phase comprising an aqueous buffer, organic solvent, and a low concentration of a non-ionic surfactant as disclosed herein provided complete and reproducible recovery of intact LNP without induction of artifacts, avoided loss by precipitation, and preserved the fragile structural integrity of delicate LNPs. In some embodiments, adsorption of LNPs is minimized, meaning the method enhances LNP % recovery relative to an SEC separation performed with a mobile phase which does not include the non-ionic surfactant. Such adsorption is believed to be due to non-ideal interactions between LNPs and the column. In some embodiments, interactions between the intact LNPs are minimized. This may be determined by a notable lack of precipitation, aggregation, and/or fouling or clogging of device components (e.g., column, detector, tubing).

### Method for Characterization of Sample Comprising Intact Lipid Nanoparticles

Accordingly, the present disclosure provides a method for characterization of a sample comprising intact lipid nanoparticles. Significantly, the disclosed method characterizes various properties of the LNP without inducing disruption of the LNP into its components. Further, the method avoids aggregation and precipitation by virtue of the specific mobile phase composition as disclosed herein below. The method generally comprises loading the sample on a chromatographic device comprising a chromatography column, flowing a mobile phase through the column to elute the intact LNPs, and detecting the eluted intact LNPs. Each component of the method is further described herein below.

### Sample

The method generally comprises loading the sample on a chromatographic device comprising a chromatography column. Loading the sample generally comprises introducing an appropriate volume of sample, neat or diluted in a suitable solvent (e.g., the mobile phase) into a chromatographic system or onto a column, each as described herein below. The terms loading, introducing, and contacting may be used interchangeably and are generally accomplished by injecting the sample, manually or in an automated fashion, into said system/column.

The nature of the sample may vary. For example, in some embodiments, the sample is a drug product or vaccine. In some embodiments, the sample is a vaccine against a viral infection, such as COVID-19.

### Size Exclusion Chromatography (SEC) and Field-Flow Fractionation (FFF)

In some embodiments, characterization comprises performing size exclusion chromatography (SEC) on the sample. SEC is a type of chromatography in which the analytes (i.e., intact LNPs as well as potential impurities) in a sample are separated or isolated on the basis of hydrodynamic radius (i.e., size). In SEC, separation occurs because of the differences in the ability of analytes to probe the volume of the porous stationary phase media. See, for example, A. M. Striegel et. al. Modern Size-Exclusion Chromatography: Practice of Gel Permeation and Gel Filtration Chromatography, 2nd Edition, Wiley, NJ, 2009. SEC is typically used for the separation of large molecules or complexes of molecules.

In some embodiments, characterization comprises performing field-flow fractionation (FFF), which is a separation technique similar to liquid chromatography. Separation according to translational diffusion coefficient or hydrodynamic size is achieved by applying a field (e.g., hydraulic) or cross-flow perpendicular to the direction of transport of the sample while the sample is pumped through a long and narrow laminar channel.

### Chromatographic Device

Chromatography, including SEC, is normally performed using a column having a packed bed of particles (“packing material”). The packing material is a separation media through which a mobile phase is flowed. The column is placed in fluid communication with a pump and a sample injector. The sample is loaded onto the column under pressure by the sample injector and the sample components and mobile phase are pushed through the column by the pump. In SEC, the components in the sample (e.g., LNPs) leave or elute from the column with the largest molecules (largest hydrodynamic radius) exiting first and the smallest molecules leaving last.

The column is placed in fluid communication with a detector, which can detect the change in the nature of the mobile phase as the mobile phase exits the column. The detector will register and record these changes as a plot, referred to as a chromatogram, which is used to determine the presence or absence of the analyte, and, in some embodiments, the concentration thereof. The time at which the analyte leaves the column (retention time) is an indication of the size of the molecule. Molecular weight of the molecules can be estimated using standard calibration curves. Examples of detectors used for SEC are, without limitation, refractive index detectors, UV detectors, light-scattering detectors, and mass spectrometers.

In some embodiments, the method comprises loading the sample on a chromatographic device comprising a chromatography column including a compartment having interior walls defining wetted surfaces and containing an SEC packing material within said compartment. A number of SEC columns of various sizes and including various packing materials are suitable for use in the method disclosed herein. Such material can be composed of one or more particles, such as one or more spherical particles. The particles are generally spherical but can be any shape useful in chromatography.

The particles have a particle size or distribution of particle sizes. Particle size may be measured, e.g., using a Beckman Coulter Multisizer 3 instrument as follows. Particles are suspended homogeneously in a 5% lithium chloride methanol solution. A greater than 70,000 particle count may be run using a 30 m aperture in the volume mode for each sample. Using the Coulter principle, volumes of particles are converted to diameter, where a particle diameter is the equivalent spherical diameter, which is the diameter of a sphere whose volume is identical to that of the particle. Particle size can also be determined by light microscopy.

In some embodiments, the particles have a size distribution in which the average (mean) diameter is from about 1 to about 50 μm, such as from about 1, about 2, about 5, about 10, or about 20, to about 30, about 40, or about 50 μm. In some embodiments, the particles have a diameter with a mean size distribution from about 1 to about 20 μm. In some embodiments, the particles have diameters ranging from between 1 to 10 μm. In some embodiments, the particles have a diameter with a mean size distribution from about 1.7 μm to about 5 μm. In some embodiments, the particles have a size distribution in which the average diameter is about 1.7 μm. In some embodiments, the particles have a size distribution in which the average diameter is about 3 μm. or about 2.5 μm.

The particles are generally porous and may be fully porous or superficially porous. Porous materials have a pore size or a distribution of pore sizes. The average pore size (pore diameter) may vary depending on the intended analyte. The pore diameter is generally selected to allow free diffusion of molecules in the sample and mobile phase so they can interact with the particles. As described in U.S. Pat. No. 5,861,110, pore diameter can be calculated from 4V/S BET, from pore volume, or from pore surface area.

In some embodiments, the porous particles have an average pore size from about 0 to about 3000 Å, or from about 40 to about 3000 Å. For example, the average pore size may be from about 40, about 50, about 60, about 70, about 80, about 90, or about 100, to about 200, about 300, about 500, about 1000, about 2000, or about 3000 Å. In some embodiments, the average pore size is from about 100 to about 500 Å. In some embodiments, the average pore size is about 125 Å. In some embodiments, the average pore size is about 200 Å. In some embodiments, the average pore size is about 250 Å. In some embodiments, the average pore size is about 270 Å. In some embodiments, the average pore size is about 450 Å. In some embodiments, the average pore size is about 900 Å. In some embodiments, the average pore size is from about 1000 to about 3000 Å, or from about 1000 to about 2000 Å. In some embodiments, the average pore size is about 1000 Å. In some embodiments, the average pore size is about 2000 Å.

Particle compositions suitable for use include, but are not limited to, inorganic materials (e.g., silica), organic material, or hybrid inorganic/organic material. Examples of suitable particle compositions are further described in US Patent Application No. 2021/0239655, U.S. Pat. Nos. 11,478,755, 11,426,707, and 7,919,177, each of which are incorporated by reference.

In some embodiments, the porous particles comprise silica, an inorganic/organic hybrid material, or a polymer. In some embodiments, the porous particles comprise silica. In some embodiments, the porous particles comprise inorganic/organic hybrid materials. In some embodiments, the porous particles comprise or are inorganic-organic hybrid ethylene bridged particles having an empirical formula of SiO₂(O_{1.5}SiCH₂CH₂SiO_{1.5})_{0.25}, referred to herein as BEH particles. Such materials may be prepared in a sol-gel synthesis by the co-condensation of 1,2-bis(triethoxysilyl)ethane (BTEE) with tetraethyl orthosilicate (TEOS). Suitable procedures are reported in Wyndham et al., Analytical Chemistry 2003, 75, 6781-6788 and U.S. Pat. No. 6,686,035, each of which is incorporated herein by reference in its entirety.

In some embodiments, the particle is a porous, diol-bonded BEH particle. In some embodiments, the particle is a diol-bonded silica particle. In some embodiments, the particle is a silica particle with a methoxy-terminated polyethylene oxide (PEO) bonding. In some embodiments, the particle is a methacrylate and polymer bead type.

In some embodiments, the packing material comprises particles with a siliceous surface, wherein the siliceous surface consists of a bonding phase formed by at least two silane compounds, wherein: one of the at least two silane compounds is a dipodal hybrid silane comprising two indirectly linked silica atoms, and one of the at least two silane compounds is a functionalized silane.

In some embodiments, the dipodal hybrid silane is selected from the group consisting of:

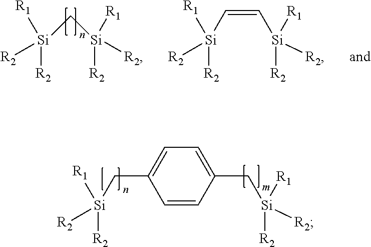

wherein:
R₁ is independently selected for each instance from chlorine, methoxy, and ethoxy;
R₂ is independently selected for each instance from alkyl, methoxy, ethoxy, and chlorine; and
n and m are each independently 1-4.

In some embodiments, the functionalized silane is selected from the group consisting of:

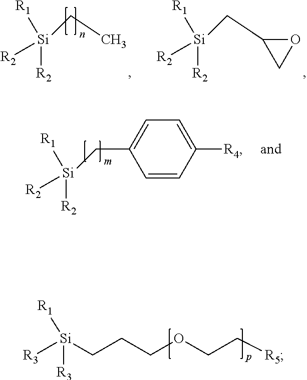

wherein:
R₁ is independently selected for each instance from chlorine, methoxy, and ethoxy;
R₂ is independently selected for each instance from alkyl, benzyl, methoxy, ethoxy, and chlorine;
R₃ is independently selected for each instance from alkyl, methoxy, ethoxy, and chlorine;
R₄ is H or benzene;
R₅ is hydroxy or methoxy;
n is 3, 7, or 17;
m is 0 or 1; and
p is an integer from 1-12.

Such siliceous packing materials are described in, for example, U.S. Provisional Application No. 63/516,197, incorporated by reference herein in its entirety.

For use in SEC, generally, the packing material will be contained in a housing having a wall defining a chamber, for example, a column having an interior for accepting the packing material. Such columns can be made of any suitable material and will have a length and a diameter.

The column material may be stainless steel, polyetheretherketone (PEEK) lined steel, titanium, or a stainless alloy. In some embodiments, the interior surfaces (i.e., walls) of the column are treated to reduce non-specific binding and enhance overall efficiency of the chromatographic device. In particular, an alkylsilyl coating or other high-performance surface is provided to limit or reduce non-specific binding of a sample with walls or interior surfaces of a column body. Without wishing to be bound by theory, it is believed that an alkylsilyl coating covering metal surfaces prevents or minimizes contact between fluids passing through the column body and the interior surfaces of the column. Typically, the alkylsilyl coating is applied to metal surfaces defining what is known as a wetted path of the column. A metal wetted path includes all surfaces formed from metal that are exposed to fluids during operation of the chromatographic column. The metal wetted path includes not only column body walls but also metal frits disposed within the column.

In general, the alkylsilyl coating is applied through a vapor deposition technique. Precursors are charged into a reactor in which the part to be coated is located. Vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating comprises a hydrophilic, non-ionic layer of polyethylene glycol silane. In another embodiment, the alkylsilyl coating is formed from one or more of the following precursor materials bis(trichlorosilyl)ethane or bis(trimethoxysilyl)ethane. Other embodiments of alkylsilyl coatings suitable for use with the present technology are described in U.S. Patent Publication No. 2019/0086371 and U.S. Application Publication No. 2022/0118443 (which are hereby incorporated by reference). For example, the alkylsilyl coating can include or consist of a C2 coating, which is the product of vapor deposited bis(trichlorosilyl)ethane or bis(trimethoxysilyl)ethane.

Column inner diameters may range from about 2.1 mm to about 7.8 mm. Column lengths may range from about 10 mm to about 300 mm. Exemplary column dimensions include, but are not limited to, 2.1×20 mm, 2.1×50 mm, 2.1×100 mm, 2.1×150 mm, 4.6×50 mm, 4.6×100 mm, 4.6×150 mm, and 4.6×300 mm. In some embodiments, the column has a bore size of about 4.6 mm inside diameter (i.d.). In some embodiments, the column has a bore size of greater than 4.6 mm i.d. In some embodiments, the column has a bore size of about 7.8 mm i.d. In some embodiments, the column has a bore size of greater than 7.8 mm i.d. In some embodiments, the column has a bore size of greater than about 4 mm i.d., greater than about 5 mm i.d., greater than about 6 mm i.d., or greater than about 7 mm i.d.

The choice of column and packing material particle, particularly with respect to pore diameter and particle size, is in part dependent on the size of the molecule to be separated and detected as would be appreciated and readily understood by one of ordinary skill in the art.

Chromatography columns suitable for use with the methods disclosed herein are compatible with any liquid chromatography system, including high-performance liquid chromatography (HPLC) and ultra-high performance liquid chromatography (UHPLC) systems, including UPLC™ systems available from Waters Corporation.

Generally, the column is connected in fluidic series to a detector, such as an ultraviolet (UV) detector or light-scattering detector. Suitable detectors are described further herein below.

### Mobile Phase

The method generally comprises flowing a mobile phase through the column packing material (e.g., SEC packing material) to elute the intact LNPs. In some embodiments, the mobile phase and, optionally the sample, are provided by a high-performance liquid chromatography (HPLC) system. The mobile phase comprises an organic solvent. As used herein, the term “organic solvent” refers to any carbon-based molecule capable of dissolving or dispersing one or more other substances (e.g., an intact LNP). Suitable solvents are generally polar and may be protic or aprotic. Suitable organic solvents include, but are not limited to, alcohols, nitriles, amides, sulfoxides, and the like. In some embodiments, the organic solvent comprises an alcohol having from 1 to 4 carbons (C1-C4 alcohol), acetonitrile, dimethyl formamide, dimethyl sulfoxide, or a combination thereof. Examples of C1-C4 alcohols include methanol, ethanol, n-propanol, isopropanol, n-butanol, isobutanol, and sec-butanol. In some embodiments, the organic solvent is isopropanol, acetonitrile, acetone, or a combination thereof. In some embodiments, the organic solvent is isopropanol.

The amount of organic solvent present in the mobile phase may vary. For example, in some embodiments, the mobile phase comprises from about 1% to about 10% by volume of the organic solvent, such as about 1, about 2, about 3, about 4, about 5, about 6, about 7, about 8, about 9, or about 10% organic solvent by volume. In some embodiments, the mobile phase comprises from about 1% to about 10% by volume of isopropanol. In some embodiments, the mobile phase comprises from about 3% to about 7%, or from about 4 to about 6% by volume of isopropanol. In some embodiments, the mobile phase comprises about 5% by volume of isopropanol.

### Buffer

The mobile phase comprises an aqueous buffer. Buffers serve to control the ionic strength and the pH of the mobile phase. Different substances may be used as buffers depending on the nature of the LNP. Non-limiting examples of suitable buffers include phosphates, tris(hydroxymethyl)aminomethane, and acetates. In some embodiments, the buffer comprises phosphate. In some embodiments, the buffer is an alkali metal phosphate. In some embodiments, the buffer is a sodium or potassium phosphate. In some embodiments, the buffer is sodium phosphate monobasic, sodium phosphate dibasic, or a combination thereof.

The concentration of the buffer may vary depending on the desired pH and ionic strength of the mobile phase. In some embodiments, the buffer is present at a concentration from about 10 to about 100 mM, such as from about 10, about 20, about 20, about 40, or about 50, to about 60, about 70, about 80, about 90, or about 100 mM.

The pH of the mobile phase may vary. In some embodiments, the pH value of the mobile phase is from about 5.0 to about 8.0. In some embodiments, the pH value of the mobile phase is from about 6.0 to about 7.5. In some embodiments, the pH is from about 6.0, or about 6.5, to about 7.0, or about 7.5. In some embodiments, the pH is about 7.0, about 7.1, about 7.2, about 7.3, about 7.4, or about 7.5.

The amount of aqueous buffer present in the mobile phase may vary. In some embodiments, the mobile phase comprises the aqueous buffer in an amount by volume from about 90 to about 99%, such as about 90, about 91, about 92, about 93, about 94, about 95, about 96, about 97, about 98, or about 99% by volume. In some embodiments, the mobile phase comprises 95% aqueous buffer by volume.

### Salts

In some embodiments, the mobile phase comprises a salt. As used herein, the term “salt” refers to an ionic compound comprising an alkali or alkaline earth metal and a halogen (e.g., fluoride, chloride, bromide, iodide). Suitable salts include, but are not limited to, sodium chloride and potassium chloride. Suitable concentrations of salts in the mobile phase range from about 1 to about 500 mM.

In some embodiments, the mobile phase comprises phosphate buffered saline comprising from about 10 to about 100 mM sodium phosphate, and further comprises sodium chloride, potassium chloride, or a combination thereof. In some embodiments, the phosphate buffered saline comprises from about 100 to about 500 mM sodium chloride and from about 1 to about 10 mM potassium chloride. In some embodiments, the phosphate buffered saline comprises from about 100 to about 500 mM potassium chloride and from about 1 to about 10 mM sodium chloride.

### Non-Ionic Surfactant

The mobile phase comprises a non-ionic surfactant. Surfactants are chemical compounds that decrease the surface tension or interfacial tension between two liquids, allowing such liquids to mix to varying degrees. Generally, surfactants are organic compounds with hydrophilic “head” groups and hydrophobic “tail” groups. The “tails” of most surfactants are fairly similar, consisting of a hydrocarbon chain, which can be branched, linear, or aromatic. The “heads” of surfactants are polar and may be charged (anionic or cationic) or neutral (non-ionic). Typically, non-ionic surfactants have covalently bonded oxygen-containing hydrophilic groups bonded to hydrophobic parent structures. Major types of non-ionic surfactants include fatty alcohol ethoxylates, alkyl phenol ethoxylates, fatty acid alkoxylates. Ethoxylates comprise a polyether chain such as ethoxylated (polyethylene oxide-like) sequences which increase the hydrophilic character of a surfactant and may further comprise polypropylene oxide chains which increase the lipophilic character of a surfactant.

In some embodiments, the non-ionic surfactant is a hydroxy-terminated polyethylene oxide-polypropylene oxide copolymer. In some embodiments, the non-ionic surfactant is a polyoxyethylene-polyoxypropylene block copolymer with the general formula (C₃H₆O·C₂H₄O)ₓ. In some embodiments, the polyoxyethylene-polyoxypropylene block copolymer has a structure:

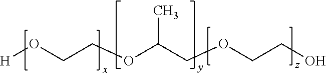

In some embodiments, the values of x, y, and z are such that the polyoxyethylene-polyoxypropylene block copolymer has a molecular weight of about 8400. One example of such a polyoxyethylene-polyoxypropylene block copolymer non-ionic surfactant is available as Pluronic® F-68 (BASF).

The concentration of non-ionic surfactant present in the mobile phase may vary. In some embodiments, the non-ionic surfactant is present in the mobile phase at a concentration sufficient to avoid denaturing the LNPs. In some embodiments, the non-ionic surfactant is present in the mobile phase at a concentration sufficient to minimize adsorption of the LNPs. In some embodiments, the non-ionic surfactant is present in the mobile phase at a concentration sufficient to minimize interactions between intact LNPs. In some embodiments, the non-ionic surfactant is present in the mobile phase at a concentration sufficient to avoid denaturing the LNPs, minimize adsorption of the LNPs (on flow path components including column hardware and packing materials), and minimize interactions between the intact LNPs.

In some embodiments, the non-ionic surfactant is present in an amount by volume from about 0.0001% to about 0.01%, based on the total volume of the mobile phase. In some embodiments, the non-ionic surfactant is present in an amount by volume of about 0.0005% to about 0.0015%, based on the total volume of the mobile phase. In some embodiments, the non-ionic surfactant is present in an amount by volume of about 0.001%.

### Flow Rate

Generally, the mobile phase is flowed through column packing material (e.g., SEC column packing material) for a period of time and at a rate sufficient to elute and/or separate various components present in the sample, including the intact LNPs. The flow rate may be determined by one of skill in the art based on scale, packing material particle size, difficulty of separation, and the like. In some embodiments, the flow rate is from about 0.05 mL/min to about 1 mL/min. In certain embodiments, the flow rate is about 0.1 mL/min.

### Temperature

The temperature at which the chromatography is performed (i.e., column temperature) may vary. In some embodiments, the column temperature is from about 20 to about 50° C., such as about 20, about 25, about 30, about 35, about 40, about 45, or about 50° C. In some embodiments, the column temperature is from about 25 to about 45° C., such as about 30° C.

### Time

The time required for the SEC separation will vary depending on many factors, but will generally be less than about 60 minutes, such as less than about 50 minutes, or less than about 40 minutes. In particular, the time will be determined by the elution time of the component(s) of interest in the sample, including but not limited to LNPs. In some embodiments, the retention time is reproducible from run to run.

### Detecting

The method comprises detecting the detecting the eluted intact LNPs. Many suitable options exist for methods of detection. In some embodiments, the detecting is performed using a refractive index detector, an ultraviolet (UV) detector, a light-scattering detector, a mass spectrometer, or combinations thereof. In some embodiments, the detecting is performed using a multi-angle light scattering (MALS) detector. In some embodiments, the detecting is performed using a UV or UV/visible detector, such as a tunable UV (TUV) detector. Numerous detectors are available; however, a specific detector is a Waters ACQUITY® UPLC® Tunable UV Detector (Waters Corporation, Milford, Mass., USA).

In some embodiments, the UV or TUV detector is tuned to measure absorbance at a wavelength in a range from about 210 nm to about 300 nm. In some embodiments, the detecting is performed at a wavelength of 230, 260, or 280 nm. In some embodiments, the detecting is performed at a wavelength of 230 or 260 nm. Said wavelengths are known in the art to detect nucleic acid molecules, including RNA. Additional detectors, such as fluorescence spectroscopy or mass spectrometry detectors can be utilized in conjunction with the disclosed methods. The detectors can be used alone or in tandem and can be further adjusted to detect molecule(s) of interest. For example, and not by way of limitation, a fluorescence detector may be utilized if the sample comprises a fluorescent molecule of interest.

All methods described herein can be performed in any suitable order unless otherwise indicated herein or otherwise clearly contradicted by context. The use of any and all examples, or exemplary language (e.g., “such as”) provided herein, is intended merely to better illuminate the materials and methods and does not pose a limitation on the scope unless otherwise claimed. No language in the specification should be construed as indicating any non-claimed element as essential to the practice of the disclosed materials and methods.

It will be readily apparent to one of ordinary skill in the relevant arts that suitable modifications and adaptations to the compositions, methods, and applications described herein can be made without departing from the scope of any embodiments or aspects thereof. The compositions and methods provided are exemplary and are not intended to limit the scope of the claimed embodiments. All of the various embodiments, aspects, and options disclosed herein can be combined in all variations. The scope of the compositions, formulations, methods, and processes described herein include all actual or potential combinations of embodiments, aspects, options, examples, and preferences herein.

Although the technology herein has been described with reference to particular embodiments, it is to be understood that these embodiments are merely illustrative of the principles and applications of the present technology. It will be apparent to those skilled in the art that various modifications and variations can be made to the method and apparatus of the present technology without departing from the spirit and scope of the technology. Thus, it is intended that the present technology include modifications and variations that are within the scope of the appended claims and their equivalents.

Reference throughout this specification to “one embodiment,” “certain embodiments,” “one or more embodiments” or “an embodiment” means that a particular feature, structure, material, or characteristic described in connection with the embodiment is included in at least one embodiment of the technology. Thus, the appearances of phrases such as “in one or more embodiments,” “in certain embodiments,” “in one embodiment” or “in an embodiment” in various places throughout this specification are not necessarily referring to the same embodiment of the technology. Furthermore, the particular features, structures, materials, or characteristics may be combined in any suitable manner in one or more embodiments. Any ranges cited herein are inclusive.

Aspects of the present technology are more fully illustrated with reference to the following examples. Before describing several exemplary embodiments of the technology, it is to be understood that the technology is not limited to the details of construction or process steps set forth in the following description. The technology is capable of other embodiments and of being practiced or being carried out in various ways. The following examples are set forth to illustrate certain aspects of the present technology and are not to be construed as limiting thereof.

### EXAMPLES

The present invention may be further illustrated by the following non-limiting examples describing the chromatographic devices and methods.

### Materials

All reagents were used as received unless otherwise noted. Those skilled in the art will recognize that equivalents of the following supplies and suppliers exist and, as such, any suppliers listed below are not to be construed as limiting.

### Example 1. SEC Chromatography of an LNP—Reference Mobile Phase (100% 2×PBS)

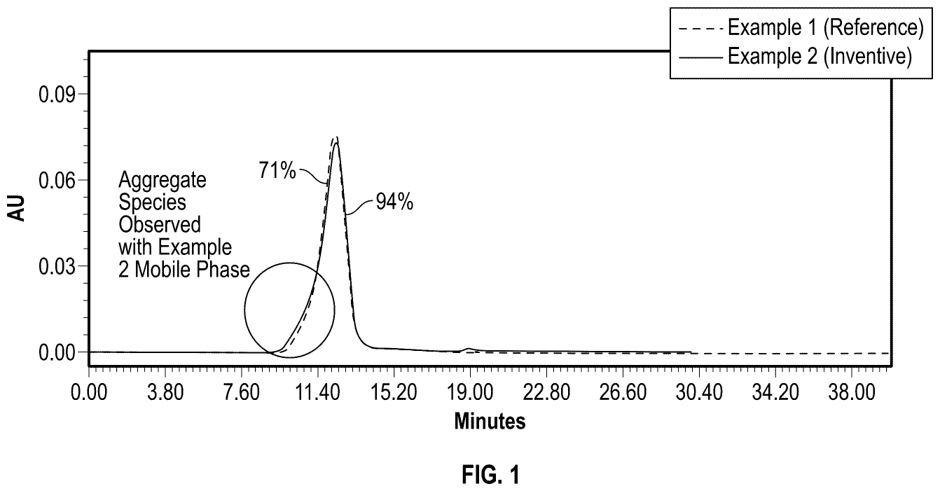

A sample of Pfizer BioNTech Covid-19 vaccine (5 microliter volume) was injected into a chromatography system (ACQUITY™ UPLC® H-Class Bio System, available from Waters Corporation). The system consisted of a QSM with 100 μL Mixer with BEH AT 5μ, 4.6×150 mm Column, TUV Detector (Flow cell: Titanium, 5 mm, 1500 nL), FTN-SM with 15 μL MP35N Needle, CH-30A heater with an Active Preheater 18.5″ and post-column tubing to the TUV (0.005″ ID×22.5″ LG MP35N Welded Tube). The mobile phase was 100% 2× phosphate buffered saline (20 mM Phosphate, 276 mM NaCl, 5.4 mM KCl, pH 7.4). That is the mobile phase of example 1 was free of organic solvent and surfactant. The column temperature was 30° C., and the flow rate was 0.1 mL/min. Optical detection through absorption was monitored at 260 nm. As illustrated in FIG. 1, the intact LNP was eluted (dashed line) with good peak shape. However, the recovery of intact LNP was only about 71%. The recovery was based on comparison of the absorbance signal obtained when the same volume of sample was injected into the same chromatography system in the absence of the column.

### Example 2. SEC Chromatography of an LNP—Inventive Mobile Phase

SEC of a sample of Pfizer BioNTech Covid-19 vaccine was performed as in Example 1 but using as the mobile phase 95% 2× phosphate buffered saline (20 mM Phosphate, 276 mM NaCl, 5.4 mM KCl, pH 7.4), 5% isopropanol (IPA), and 0.001% polyoxyethylene-polyoxypropylene block copolymer (i.e., poloxamer available as Pluronic® F-68 surfactant, from BASF. All other parameters remained the same. Using the inventive mobile phase, the recovery of intact LNP was about 94%, and aggregate species were observed in the chromatogram (FIG. 1; solid line). Table 1 provides absorbance unit area values at wavelengths of 280 nm, 260 nm and 230 nm. With reference to Table 1, the 280 nm wavelength showed reduced sensitivity, and the ratio of absorbance area at 260 nm to absorbance area at 230 nm was approximately 0.8 (based on average of two separate injections for each wavelength). Accordingly, when monitored at a wavelength of 230 nm, the calculated LNP % recovery was 101.4%.

Further, the chromatogram of FIG. 1 shows partial fronting, indicating slight differences in elution time of components of the sample. This demonstrates the presence of higher molecular weight/hydrodynamic radius species in the sample and their separation by size, with the larger species eluting earlier.

TABLE 1
Absorbance unit areas vs. monitoring wavelength

| | Wavelength | | | | | |
|---|---|---|---|---|---|---|
| | 230 nm | | 260 nm | | 280 nm | |
| | Inj#1 | Inj#2 | Inj#1 | Inj#2 | Inj#1 | Inj#2 |
| Area | 8895558 | 9158464 | 7142200 | 6916008 | 4533409 | 4582356 |

| | | | |
|---|---|---|---|
| Average | 9027011 | 7029104 | 4557883 |

### Example 3. SEC Chromatography of an LNP—Reference Mobile Phase (95% 2×PBS and 5% IPA)

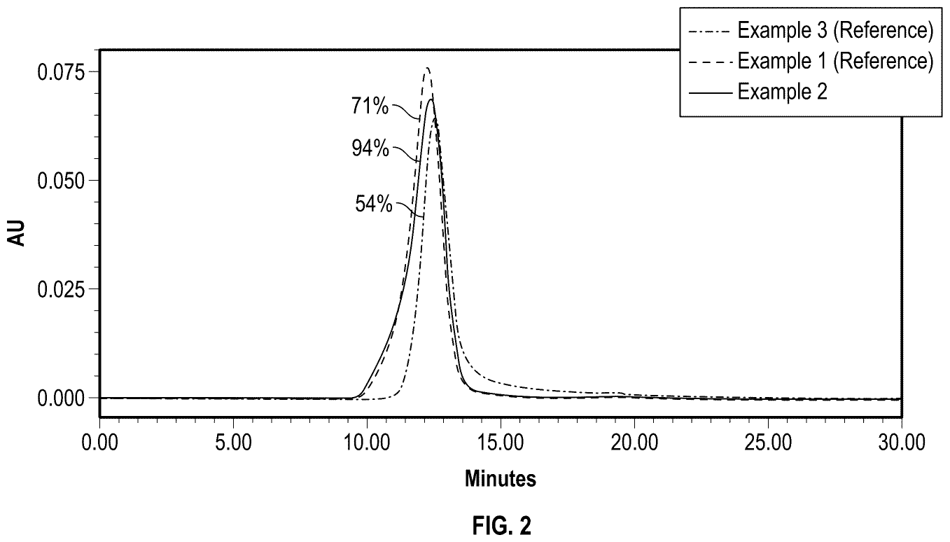

SEC of a sample of Pfizer BioNTech Covid-19 vaccine was performed as in Example 1 but using as the mobile phase 95% 2× phosphate buffered saline (20 mM Phosphate, 276 mM NaCl, 5.4 mM KCl, pH 7.4) with 5% isopropanol. As illustrated in FIG. 2, the intact LNP was eluted (dotted line) with good peak shape. However, the recovery of intact LNP was only about 54%.

Overall, the results disclosed herein demonstrate that the inventive mobile phase according to Example 2 keeps the LNPs in solution, minimizes or eliminates interactions between the LNPs, and minimizes or eliminates interactions between the LNPs and the fluidic path of the system (e.g., column walls, detector, tubing, and the like). In contrast, the low recovery for the reference mobile phases of Examples 1 and 3 suggests a failure to avoid the foregoing interactions and/or to maintain full solution of the LNPs.

### Example 4. SEC-MALS Chromatography of an LNP—Inventive Mobile Phase

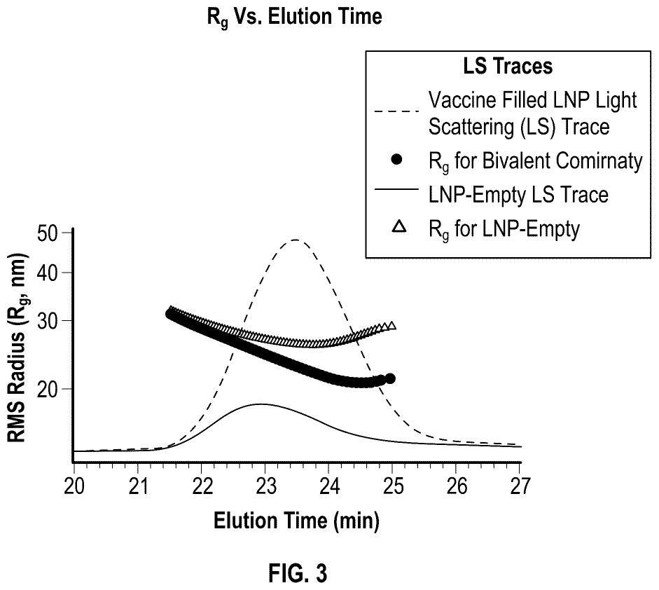

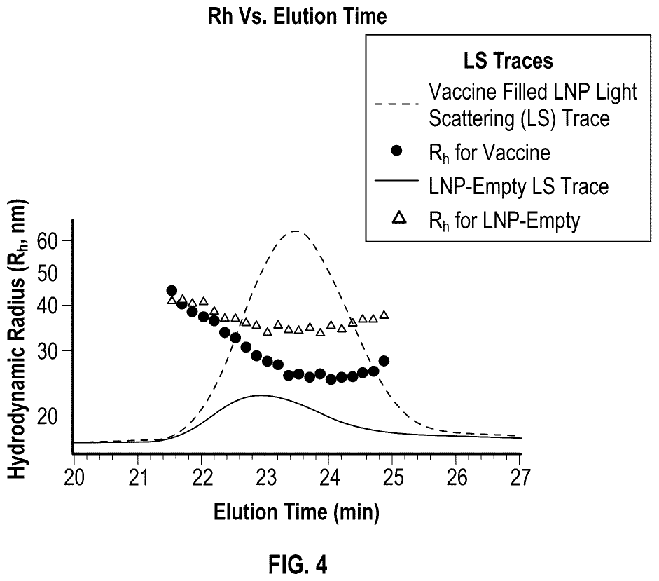

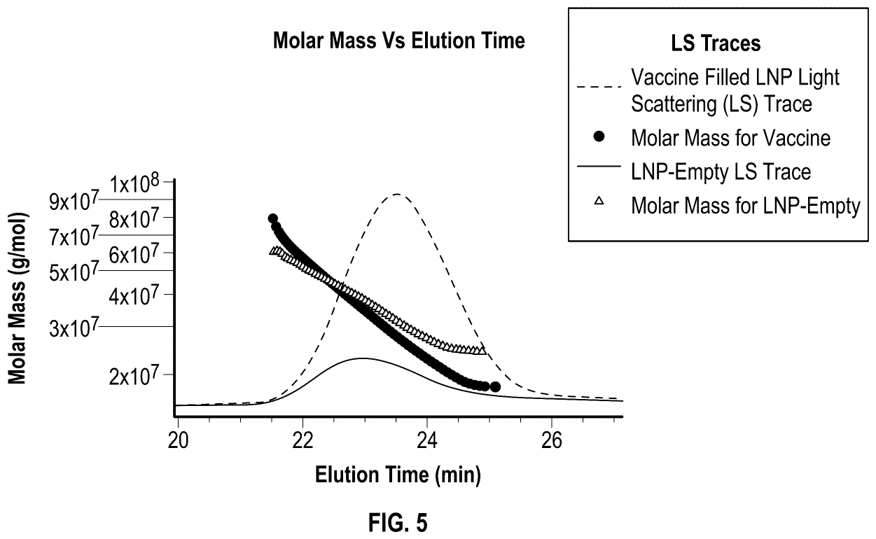

SEC of a sample of Pfizer BioNTech Covid-19 vaccine was performed as in Example 2. The eluent of the SEC column was then analyzed using a multi-angle-light-scattering (MALS) detector (Wyatt DAWN™ MALS detector with a WyattQES Embedded Online Dynamic Light Scattering (DLS) Module, available from Wyatt Technologies, Santa Barbara, CA). Thus the configuration of the system was arranged as follows: r-QSM→r-FTN(30-channel)→TUV(2489)→DAWN(NEON)→OptiLab→Waste. FIG. 3, FIG. 4, and FIG. 5 compare the measured radius of gyration (R_{g}), hydrodynamic radius (Rₕ), and molar mass respectively as measured by MALS for each of the vaccine (i.e., LNP containing COVID vaccine) and empty LNPs sample components (i.e., no nucleic acid within the particles) as a function of elution time. Light scattering (LS) traces for the LNPs (vaccine filled, and empty) are shown on a chromatographic time scale (dashed line of the vaccine filled and solid line for the empty LNP samples). Notably, the light scattering signal resulting from the vaccine was higher than that for the empty LNP.

In FIG. 3 light scattering (LS) traces for the LNPs (Vaccine filled and empty) are shown on chromatographic time scale (dashed line of the vaccine filled and solid line for the empty LNP samples). Rg values measured by software at each elution time point are shown as horizontal trails on each LS chromatogram (circle for the vaccine; triangle for empty LNP). The Rg values represent the change in the geometric radius or radius of gyration measurements across each peak with larger species (left) elution early. The R_{g} values measured in FIG. 3 range from 21.2 nm to 31.1 nm for the vaccine (mean R_{g}=23 nm) and from 29.0 nm to 31.9 nm (mean R_{g}=27 nm) for the empty LNP.

In FIG. 4 light scattering (LS) traces for the LNPs (vaccine filled, and empty) are shown on a chromatographic time scale (dashed line of the vaccine filled and solid line for the empty LNP samples). Rₕ values measured by software at each elution time point are shown as horizontal trails on each LS chromatogram (circle for the vaccine; triangle for empty LNP). The Rₕ values represent the change in the hydrodynamic radius measurements across each peak with larger species (left) elution early. The Rₕ values measured in FIG. 4 range from 28.1 nm to 42.7 nm (mean Rₕ=33 nm) for the vaccine and 37.5 nm to 42.1 nm (mean=37 nm) for the empty LNP.

In FIG. 5 light scattering (LS) traces for the LNPs (vaccine filled, and empty) are shown on a chromatographic time scale (dashed line of the vaccine filled and solid line for the empty LNP samples). Molar mass values measured by software at each elution time point are shown as horizontal trails on each LS chromatogram (circle for the vaccine; triangle for empty LNP). The molar mass values represent the change in the molar mass measurements across each peak with larger species (left) elution early. The molar mass values measured in FIG. 5 range from 18.1 MDa to 72.5 MDa for the vaccine (mean value 27 MDa) and from 24.2 MDA to 56.1 MDa for the empty LNP (mean value of 34 MDa).

Overall, the results disclosed herein demonstrate that the inventive mobile phase according to Example 2 is capable of down-stream analysis with non-absorbance based detectors including MALS detectors.

1. A method for characterization of a sample comprising intact lipid nanoparticles (LNPs), wherein the characterization comprises performing size exclusion chromatography (SEC) on the sample, the method comprising:
a) loading the sample on a chromatography column including a compartment having interior walls defining wetted surfaces and containing a column packing material configured for SEC within said compartment;
b) flowing a mobile phase through the column packing material to elute the intact LNPs, the mobile phase comprising an organic solvent, an aqueous buffer, and a non-ionic surfactant, wherein the non-ionic surfactant is present in the mobile phase at a concentration sufficient to avoid denaturing the LNPs, minimize adsorption of the LNPs, and minimize interactions between the intact LNPs; and
c) detecting the eluted intact LNPs.

2. The method of claim 1, wherein the non-ionic surfactant is present in an amount by volume from about 0.0001% to about 1%, based on the total volume of the mobile phase.

3. (canceled)

4. The method of claim 1, wherein the non-ionic surfactant is present in an amount by volume from about 0.0005% to about 0.0015%, based on the total volume of the mobile phase.

5. The method of claim 1, wherein the non-ionic surfactant is a hydroxy-terminated polyethylene oxide-polypropylene oxide copolymer.

6. The method of claim 5, wherein the non-ionic surfactant is a polyoxyethylene-polyoxypropylene block copolymer with the general formula (C₃H₆O·C₂H₄O)ₓ having a molecular weight of about 8400.

7. The method of claim 1, wherein the organic solvent is isopropanol, acetonitrile, acetone, or a combination thereof.

8. The method of claim 1, wherein the organic solvent is isopropanol.

9. The method of claim 8, wherein the isopropanol is present in the mobile phase at a concentration from about 1% to about 10% (v/v).

10. The method of claim 1, wherein the aqueous buffer is phosphate buffered saline having a pH of about 7.4.

11. The method of claim 10, wherein the phosphate buffered saline and comprises from about 10 to about 100 mM sodium phosphate.

12. (canceled)

13. The method of claim 12, wherein the phosphate buffered saline comprises:
from about 100 to about 500 mM sodium chloride, and from about 1 to about 10 mM potassium chloride; or
from about 100 to about 500 mM potassium chloride, and from about 1 to about 10 mM sodium chloride.

14. The method of claim 1, wherein the mobile phase comprises the aqueous buffer in an amount by volume from about 90 to about 99%.

15. The method of claim 1, wherein the mobile phase comprises about 95% phosphate buffered saline, about 5% isopropanol, and about 0.001% of hydroxy-terminated polyoxyethylene-polyoxypropylene triblock co-polymer.

16. The method of claim 1, wherein the detecting is performed with a dual wavelength ultraviolet/visible detector, an evaporative light scattering detector, or a multi-angle light scattering (MALS) detector.

17. The method of claim 1, wherein the detecting is performed with a dual wavelength ultraviolet/visible detector at a wavelength of 230, 260, or 280 nm.

18. The method of claim 1, wherein at least a portion of the interior walls defining wetted surfaces comprises a coating configured to reduce hydrophobic secondary interactions between the intact LNPs and said interior walls.

19. The method of claim 1, wherein the packing material configured for SEC comprises diol-bonded porous particles having a particle size from about 1 μm to about 10 μm and an average pore diameter in a range from about 100 Å to about 5000 Å.

20. The method of claim 1, wherein the packing material configured for SEC comprises particles with a siliceous surface, wherein the siliceous surface consists of a bonding phase formed by at least two silane compounds, wherein:
one of the at least two silane compounds is a dipodal hybrid silane comprising two indirectly linked silica atoms, and
one of the at least two silane compounds is a functionalized silane.

21. The method of claim 20, wherein the dipodal hybrid silane is selected from the group consisting of: 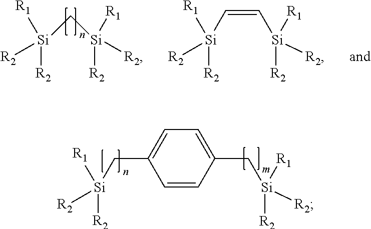
wherein:
R₁ is independently selected for each instance from chlorine, methoxy, and ethoxy;
R₂ is independently selected for each instance from alkyl, methoxy, ethoxy, and chlorine; and
n and m are each independently 1-4.

22. The method of claim 20, wherein the functionalized silane is selected from the group consisting of: 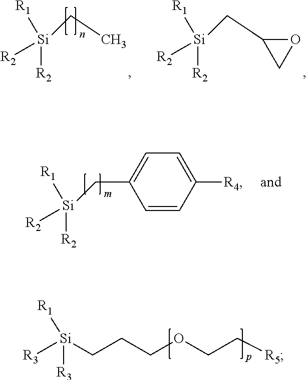
wherein:
R₁ is independently selected for each instance from chlorine, methoxy, and ethoxy;
R₂ is independently selected for each instance from alkyl, benzyl, methoxy, ethoxy, and chlorine;
R₃ is independently selected for each instance from alkyl, methoxy, ethoxy, and chlorine;
R₄ is H or benzene;
R₅ is hydroxy or methoxy;
n is 3, 7, or 17;
m is 0 or 1; and
p is an integer from 1-12.

23. (canceled)

24. (canceled)
